(19) United States
(12) Patent Application Publication
(10) Pub. No.: US 2026/0034103 A1
(43) Pub. Date: Feb. 5, 2026

(54) COMPOSITIONS AND METHODS FOR IMPROVING MEMORY AND COGNITION

(71) Applicant: SIRTSEI PHARMACEUTICALS, INC., Morrisville, NC (US)

(72) Inventors: Michael Friedrich Ackermann, Holly Springs, NC (US); Stephen E. Butts, Apex, NC (US); Gregory Cooksey Rigdon, Wake Forest, NC (US)

(21) Appl. No.: 18/996,613

(22) PCT Filed: Jul. 28, 2023
(86) PCT No.: PCT/US2023/071185

§ 371 Date: Jan. 17, 2025

Related U.S. Application Data
(60) Provisional application No. 63/393,049, filed on Jul. 28, 2022.

(51) Int. Cl. A61K 31/4245; A61P 25/28

(52) U.S. Cl. CPC A61K 31/4245; A61P 25/28

(57) ABSTRACT

The present invention relates to methods of improving memory function, cognition function, and learning function by administering a compound of Formula I to a subject in need thereof. The invention further relates to methods of delaying or slowing the loss of memory function, cognition function, and learning function by administering a compound of Formula 1 to a subject in need thereof.

### STATEMENT OF PRIORITY

This application claims the benefit of U.S. Provisional Application Ser. No. 63/393,049, filed Jul. 28, 2022, the entire contents of which are incorporated by reference herein.

### FIELD OF THE INVENTION

The present invention relates to methods of improving memory function, cognition function, and learning function by administering a compound of Formula 1 to a subject in need thereof. The invention further relates to methods of delaying or slowing the loss of memory function, cognition function, and learning function by administering a compound of Formula 1 to a subject in need thereof.

### BACKGROUND OF THE INVENTION

Brain function, particularly memory and cognition, is critical for conducting daily life for people of all ages. Tasks such as learning and job performance require optimal memory and cognition.

Memory, cognition, and learning function naturally decine with age due to brain atrophy and shrinkage. Declines may also be associated with neurological disorders and brain injuries.

Griseofulvin derivatives have been developed that have anti-inflammatory activity and have been disclosed for use in treating inflammatory diseases, including central inflammatory disease such as neurodegenerative diseases. See WO 2017/170623 and WO 2019/065928.

There is a need in the art for effective treatments to improve memory, cognition function and learning function or delay the loss of memory, cognition function and learning function.

### SUMMARY OF THE INVENTION

The present invention is based on the determination that the compounds of the invention act to improve physiological characteristics associated with cognition and memory, such as enhancing neuronal transmission in the hippocampus, improving scopolamine-induced cognitive impairment, ameliorating cognitive deficits involved in NMDA receptor hypofunction, and increasing long-term potentiation. Further, the compounds of the invention have been shown in clinical studies to improve concentration and decision making.

Thus, one aspect of the invention relates to a method of improving memory function, cognition function, and/or learning function in a subject in need thereof, comprising administering to the subject a therapeutically effective amount of the compound of Formula 1 or a pharmaceutically acceptable salt thereof:

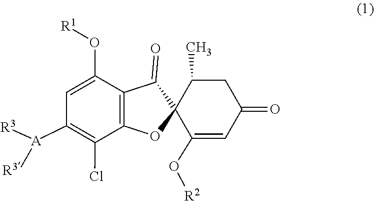

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH=, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group, a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
substituent group Y is a C1-C6 alkyl group, a C1-C6 alkoxy group, a halogen atom, or a hydroxy group,
thereby improving memory function, cognition function, and/or learning function in the subject.

Another aspect of the invention relates to a method of delaying or slowing a decrease in memory function, cognition function, and/or learning function in a subject in need thereof, comprising administering to the subject a therapeutically effective amount of a compound of Formula 1 or a pharmaceutically acceptable salt thereof:

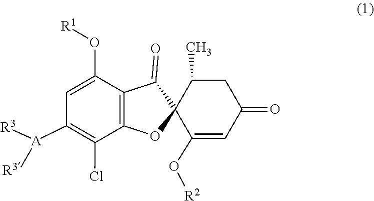

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH=, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group, a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group, a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
substituent group Y is a C1-C6 alkyl group, a C1-C6 alkoxy group, a halogen atom, or a hydroxy group,
thereby delaying or slowing a decrease in memory function, cognition function, and/or learning function in the subject.

The invention additionally relates to use of the compounds of the invention to improve memory function, cognition function, and/or learning function.

The invention further relates to use of the compounds of the invention to delay or slow a decrease in memory function, cognition function, and/or learning function.

The invention additionally relates to use of the compounds of the invention in the preparation of a medicament to improve memory function, cognition function, and/or learning function.

The invention further relates to use of the compounds of the invention in the preparation of a medicament to delay or slow a decrease in memory function, cognition function, and/or learning function.

These and other aspects of the invention are set forth in more detail in the description of the invention below.

### BRIEF DESCRIPTION OF THE DRAWINGS

FIG. 1 shows paired-pulse ratio (PPF index is defined as slopes of fEPSP2/fEPSP1) at interstimulus intervals of 50-400 ms from rats after perfusion with compound 1 (10 nM) or vehicle (0.01% DMSO) or positive control (dopamine 30 μM). Response is Stratum radiatum of area CA1 of the hippocampus with Schaffer collateral stimulation. Statistical differences correspond to the ones observed between Compound 1 10 nM and Vehicle groups.

FIG. 2 shows paired-pulse ratio at interstimulus intervals of 50-400 ms from rats after perfusion with compound 1 (100 nM) or vehicle (0.01% DMSO) or positive control (dopamine 30 μM) Response is Stratum radiatum of area CA1 with Schaffer collateral stimulation. Statistical differences correspond to the ones observed between Compound 1 100 nM and Vehicle groups.

FIG. 3 shows the time course of the average slope of elicited field responses following short-lasting long-term potentiation (S-LTP) induction by a single 1-sec train of 100-Hz stimulation at hippocampal CA1 synapses from rats after perfusion with compound 1 (10 nM), vehicle (0.01% DMSO), or positive control (dopamine 30 μM). Time-point 0 represents delivery of HFS. Slopes of fEPSP are normalized to baseline and plotted against time.

FIG. 4 shows total exploration time in the acquisition trial in novel object recognition test in rats. Total exploration time of two identical objects in the acquisition trial was measured manually and indicated as the mean±SEM (n=8). No statistical difference was found in the total exploration time among the different groups by one-way ANOVA (P=0.115).

FIG. 5 shows the effect of compound 1 in the novel object recognition test in rats. NDI was calculated using the following equation: novel object interaction time/total interaction time×100(%). Data are represented as the mean±SEM (n=8). Significant difference from control group was indicated by ***P<0.001 (Student's t-test). Significant differences from scopolamine-treated vehicle group were indicated by ##P<0.01 (Dunnett test). Significant difference from scopolamine-treated vehicle group were indicated by †††P<0.001 (Student's t-test).

FIG. 6 shows exploration time for familiar and novel objects in the retention trial in novel objection recognition test in rats. Exploration time of each object in retention trial was measured manually and presented as the mean±SEM (n=8). Significant differences from familiar object interaction were indicated by **P<0.01, *P<0.05 (paired t-test).

FIG. 7 shows total exploration time in the acquisition trial in novel object recognition test in rats. The total exploration time of two identical objects in the acquisition trial was measured manually and indicated as the mean±SEM (n=10). No statistical difference was found in the total exploration time among the different groups by one-way ANOVA (P=0.340).

FIG. 8 shows the effect of compound 1 in the novel object recognition test in rats. NDI was calculated using the following equation: novel object interaction time/total interaction time×100(%). Data are represented as the mean±SEM (n=10). Significant difference from control group was indicated by ***P<0.001 (Student's t-test). Significant differences from PCP-treated group were indicated by ##P<0.01, #P<0.05 (Dunnett test). Significant difference from PCP-treated group were indicated by ††P<0.01 (Student's t-test).

FIG. 9 shows exploration time for familiar and novel objects in the retention trial in novel objection recognition test in rats. Exploration time of each object in retention trial was measured manually and presented as the mean±SEM (n=10). Significant differences from familiar object interaction were indicated by ***P<0.001, **P<0.01 (paired t-test).

FIG. 10 shows the time course of the average amplitude of Stratum radiatum fEPSP following long term potentiation (LTP) induction by a 100-Hz stimulation (10 bursts of 10 stimuli, 0.1 msec stimulus duration, 10 sec interburst interval) at Schaffer collateral/commissural pathway from rats after administration of compound 1 (0.1 mg/kg, 0.01 mg/kg, 0.001 mg/kg) or vehicle (0.01% DMSO) or positive control (memantine 10 mg/kg). Amplitudes of fEPSP are normalized to baseline and plotted against time.

FIG. 11 shows average Statum radiatum fEPSP amplitude during the 3 baseline sessions (Basseline 1, Baseline 2, Baseline 3), 60 minutes following HFT delivery (Post HFT 0-60 min) and 24 hours after HFT delivery (Post HFT 24 h) from rats after administration of compound 1 (0.1 mg/kg, 0.01 mg/kg, 0.001 mg/kg) or vehicle (0.01% DMSO) or positive control (memantine 10 mg/kg).

FIG. 12 shows the results from Quick Inventory of Depressive Symptomatology—Self Reporting (QIDS-SR) Item #10 Concentration/Decision Making.

FIG. 13 shows the results from Hamilton Depression Rating Scale (Ham-D) Item #17 Retardation.

FIG. 14 shows the results from Montgomery-Asberg Depression Rating Scale (MADRS) Item #6 Concentration Difficulties.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, 5%, 1%, 0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, sterile pyrogen-free water and sterile pyrogen-free physiological saline solution.

“Concurrently” means sufficiently close in time to produce a combined effect (that is, concurrently can be simultaneously, or it can be two or more events occurring within a short time period before or after each other). In some embodiments, the administration of two or more compounds “concurrently” means that the two compounds are administered closely enough in time that the presence of one alters the biological effects of the other. The two compounds can be administered in the same or different formulations or sequentially. Concurrent administration can be carried out by mixing the compounds prior to administration, or by administering the compounds in two different formulations, for example, at the same point in time but at different anatomic sites or using different routes of administration.

Memory is the process by which data or information is encoded, stored, and retrieved when needed. The memory processing system in the brain is made up of a sensory processor, short-term (or working) memory, and long-term memory. Memory function, as used herein, refers to the level of memory that can be quantified on a memory performance test. Increases and decreases in memory function can be quantitated by repeated use of such tests.

Cognition refers to the mental action or process of acquiring knowledge and understanding through thought, experience, and the senses. It encompasses all aspects of intellectual functions and processes including perception, attention, thought, intelligence, the formation of knowledge, memory, and working memory, judgement and evaluation, reasoning and computation, problem solving, and decision making, comprehension, and production of language. Cognition function, as used herein, refers to the level of cognition that can be quantified on a cognition performance test. Increases and decreases in cognition function can be quantitated by repeated use of such tests.

Learning is the process of acquiring new understanding, knowledge, behaviors, skills, values, and preferences. Learning function, as used herein, refers to the level of learning that can be quantified on a learning performance test. Increases and decreases in learning function can be quantitated by repeated use of such tests.

One aspect of the invention relates to a method of improving memory function, cognition function, and/or learning function in a subject in need thereof, comprising administering to the subject a therapeutically effective amount of the compound of Formula 1 or a pharmaceutically acceptable salt thereof:

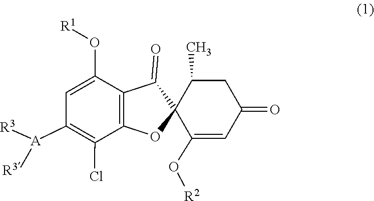

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH=, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, substituent group Y is a C1-C6 alkyl group, a C1-C6 alkoxy group, a halogen atom, or a hydroxy group,
thereby improving memory function, cognition function, and/or learning function in the subject.

A further aspect of the invention relates to a method of delaying or slowing a decrease in memory function, cognition function, and/or learning function in a subject in need thereof, comprising administering to the subject a therapeutically effective amount of the compound of Formula 1 or a pharmaceutically acceptable salt thereof:

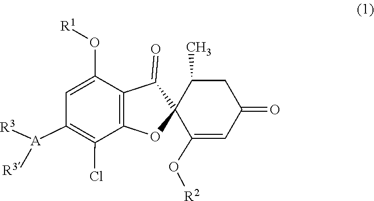

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a CT-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH=, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group, a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group, a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group, a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group, a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, substituent group Y is a C1-C6 alkyl group, a C1-C6 alkoxy group, a halogen atom, or a hydroxy group,
thereby delaying or slowing the decrease in memory function, cognition function, and/or learning function in the subject.

The methods of the invention may be used for any subject that is in need of improving memory function, cognition function, and/or learning function or in need of delaying or slowing the decrease in memory function, cognition function, and/or learning function. In some embodiments, the subject has normal (e.g., average) memory function, cognition function, and/or learning function relative to the general population. The subject may be one that is looking for improved memory, cognition, and/or learning to enhance achievement in school, at a job, or other situations. The subject may be one that does not have a factor, such as a neurological disorder or brain injury, that is decreasing or has decreased memory function, cognition function, and/or learning function. The subject may be one that does not have a decline in memory function, cognition function, and/or learning function due to ageing.

In some embodiments, the subject has reduced memory function, cognition function, and/or learning function relative to the general population. The reduction may be due to aging. In some embodiments, the reduction is not due to ageing. The reduction may be due to factors such as a neurological disorder or brain injury. Examples of factors associated with reduced memory, cognition, and/or learning include, without limitation, Alzheimer's disease, attention deficit disorder, dementia with Lewy bodies disease, early onset dementia, epilepsy-related cognitive dysfunction, fronto-temporal dementia, mild cognitive impairment, normal pressure hydrocephalus, Parkinson's disease-related cognitive dysfunction, posterior cortical atrophy, primary progressive aphasia, stroke-related cognitive dysfunction, traumatic brain injury, other psychiatric and/or neurologic disorder-related cognitive impairments (e.g., multiple sclerosis, schizophrenia, amyotrophic lateral sclerosis, Huntington's disease, chronic depression), and chemotherapy-related cognitive impairment (chemo-brain).

Memory function, cognition function, and learning function can be measured by tests well known in the art, including but not limited to, the Stroop test, Rey Auditory Verbal Learning Test (RAVLT), Wechsler Adult Intelligence Scale (WAIS) test, Memex 100, Mini-Mental Status, Activities of Daily Living, Clinical Dementia Rating, or a combination thereof. The tests may be repeated to quantitate changes in memory, cognition, and learning over time.

In some embodiments, the methods of the invention may improve memory function, cognition function, and/or learning function by at least 5% relative to the baseline before treatment, e.g., at least 10%, 20%, 30%, 40%, 50%, 60%, 70%, 80%, 90%, 100%, or more. In some embodiments, the methods of the invention may slow loss of memory function, cognition function, and/or learning function by at least 5% relative to subjects that have not received the treatment, e.g., at least 10%, 20%, 30%, 40%, 50%, 60%, 70%, 80%, 90%, 100%, or more. In some embodiments, the methods of the invention may delay loss of memory function, cognition function, and/or learning function by at least one month relative to subjects that have not received the treatment, e.g., at least three months, six months, one year, or more.

In some embodiments, the compound of Formula 1 is any compound selected from the following group:
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl]spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5]dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione; or
(2S,5′R)-7-chloro-3′,4-dimethoxy-5′-methyl-6-(3-pyridyl) spiro[benzofuran-2,4′-cyclohex-2-ene]-1′,3-dione.

In some embodiments, the compound of Formula 1 is a compound of Formula 1′ or a pharmacologically acceptable salt thereof:

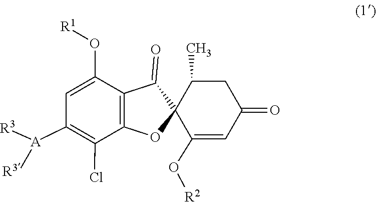

wherein:
R¹ is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different
one to two substituents selected from the substituent group X.
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, or a single bond, wherein when it is a single bond, one or the other of R³ and R^{3′} is not present,
R³ and R^{3′} are each independently a hydrogen atom, a halogen atom, a cyano group, a hydroxy group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X,
or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from the substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, and
substituent group Y is a C1-C6 alkyl group, a C1-C6 alkoxy group, a halogen atom, or a hydroxy group.

In some embodiments of the compound of Formula 1 or Formula 1′, R¹ is a C1-C6 alkyl group, R² is a C1-C6 alkyl group, A is a 5-membered aromatic heterocyclic ring, and R³ and R^{3′} are each independently a hydrogen or a C1-C6 alkyl group.

In some embodiments of the compound of Formula 1 or Formula 1′, R¹ is a methyl group, an ethyl group, or a hydroxyethyl group.

In some embodiments of the compound of Formula 1 or Formula 1′, R² is a methyl group.

In some embodiments of the compound of Formula 1 or Formula 1′, A is a 5-membered aromatic heterocyclic ring, R³ is a methyl group, an ethyl group, a hydroxy C1-C3 alkyl group, or a methoxy C1-C3 alkyl group, and R^{3′} is a hydrogen atom.

In some embodiments, the compound of Formula 1 is a compound of a Formula 1″ or a pharmacologically acceptable salt thereof:

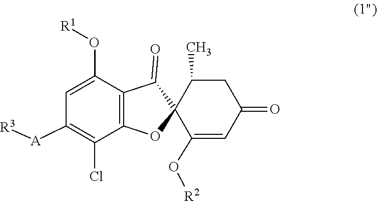

wherein R¹ is a methyl group or an ethyl group;
R² is a methyl group;
A is any ring selected from the following group:

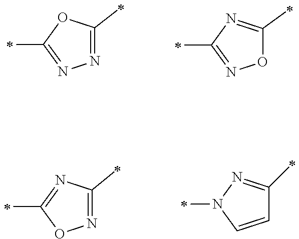

indicates a binding group; and
R³ is a methyl group or an ethyl group.

In some embodiments, the compound of Formula 1′ is any compound selected from the following group:
(2S,5′R)-7-chloro-6-(2-hydroxyethoxy)-3′,4-dimethoxy-5′-methyl-spiro[benzofuran-2,4′-cyclohex-2-ene]-1′,3-dione;
(2S,5′R)-7-chloro-3′,4-dimethoxy-6-(2-methoxyethoxy)-5′-methyl-spiro[benzofuran-2,4′-cyclohex-2-ene]-1′,3-dione;
(2S,5′R)-7-chloro-3′,4-dimethoxy-5′-methyl-6-(1-methylpyrazol-3-yl) spiro[benzofuran-2,4′-cyclohex-2-ene]-1′,3-dione;
(2S,5′R)-7-chloro-6-(1-ethylpyrazol-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-(3-methyl-1,2,4-oxadiazol-5-yl) spiro[benzofuran-2,4′-cyclohex-2-ene]-1′,3-dione;
(2S,5′R)-7-chloro-3′,4-dimethoxy-5′-methyl-6-(5-methyl)-1,2,4-oxadiazol-3-yl) spiro[benzofuran-2,4′-cyclohex-2-ene]-1′,3-dione;
(2S,5′R)-7-chloro-6-[5-(1-hydroxy-1-methyl-ethyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-6-[5-[(1S)-1-hydroxyethyl]-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-6-[5-[(1R)-1-hydroxyethyl]-1,3,4-oxadiazol-2-yl]-3′,4-dimethoxy-5′-methyl-spiro[benzofuran-2,4′-cyclohex-2- ene]-1′,3-dione;
(2S,5′R)-7-chloro-4-ethoxy-6-[5-(1-hydroxy-1-methyl-ethyl)-1,3,4-oxadiazol-2-yl]-3′-methoxy-5′-methyl-spiro[benzofuran-2,4′-cyclohex-2-ene]-1′,3-dione;
(2S,5′R)-7-chloro-4-ethoxy-6-[5-[(1S)-1-hydroxyethyl]-1,3,4-oxadiazol-2-yl]-3′-methoxy-5′-methyl-spiro[benzofuran-2,4′-cyclohex-2-ene]-1′,3-dione;
(2S,5′R)-7-chloro-6-[3-(1-hydroxyethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(2-hydroxyethoxy)-3′-methoxy-5′-methyl-6-(3-methyl-1,2,4-oxadiazol-5-yl) spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,2,4-oxadiazol-3-yl) spiro[benzofuran-2,4′-cyclohex-2-ene]-1′,3-dione;
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl]spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′
methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2- ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5]dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione;
(2S,5′R)-7-chloro-3′,4-dimethoxy-5′-methyl-6-(3-pyridyl) spiro[benzofuran-2,4′-cyclohex-2-ene]-1′,3-dione; or
(2S,5′R)-7-chloro-3′,4,6-trimethoxy-5′-methyl-spiro[benzofuran-2,4′-cyclohex-2-ene]-1′,3-dione.

In one embodiment, the compound is (2S,5′R)-7-chloro-6-(1-ethylpyrazol-3-yl)-3′,4-dimethoxy-5′-methyl-spiro[benzofuran-2,4′-cyclohex-2-ene]-1′,3-dione or a pharmacologically acceptable salt thereof.

In one embodiment, the compound is (2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione or a pharmacologically acceptable salt thereof.

In one embodiment, the compound is (2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione or a pharmacologically acceptable salt thereof.

In one embodiment, the compound is (2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2- ene]-1′,3-dione or a pharmacologically acceptable salt thereof.

In one embodiment, the compound is (2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione or a pharmacologically acceptable salt thereof.

In certain embodiments, the 5-membered aromatic heterocyclic ring for A is the same as described above, but more preferably, it represents the following 5-membered ring. (It should be noted that in this case, R^{3′} is not present.)

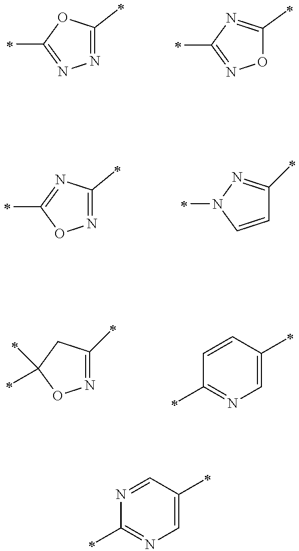

wherein * indicates a binding group.

In the present specification, the “5-membered aromatic heterocyclic ring” is a monocyclic 5-membered aromatic heterocyclic ring containing one to four atoms selected from the group consisting of a nitrogen atom, an oxygen atom, and a sulfur atom. For example, rings such as those shown below are included.

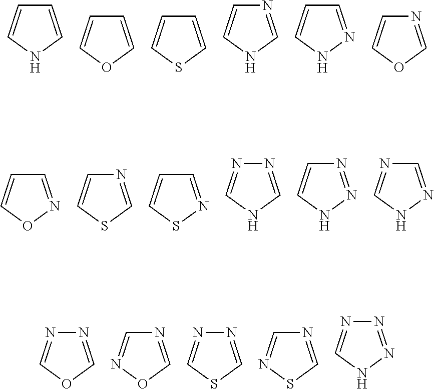

In the present specification, the “6-membered aromatic heterocyclic ring” is a monocyclic 6-membered aromatic heterocyclic ring containing one to four atoms selected from the group consisting of a nitrogen atom, an oxygen atom, and a sulfur atom. For example, rings such as those shown below are included.

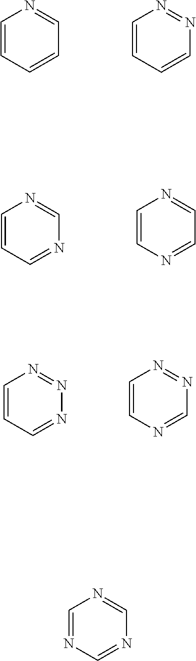

In the present specification, the “8-10 membered condensed aromatic heterocyclic ring” is an 8-10 membered condensed aromatic heterocyclic ring containing one to four atoms selected from the group consisting of a nitrogen atom, an oxygen atom, and a sulfur atom. For example, rings such as those shown below are included.

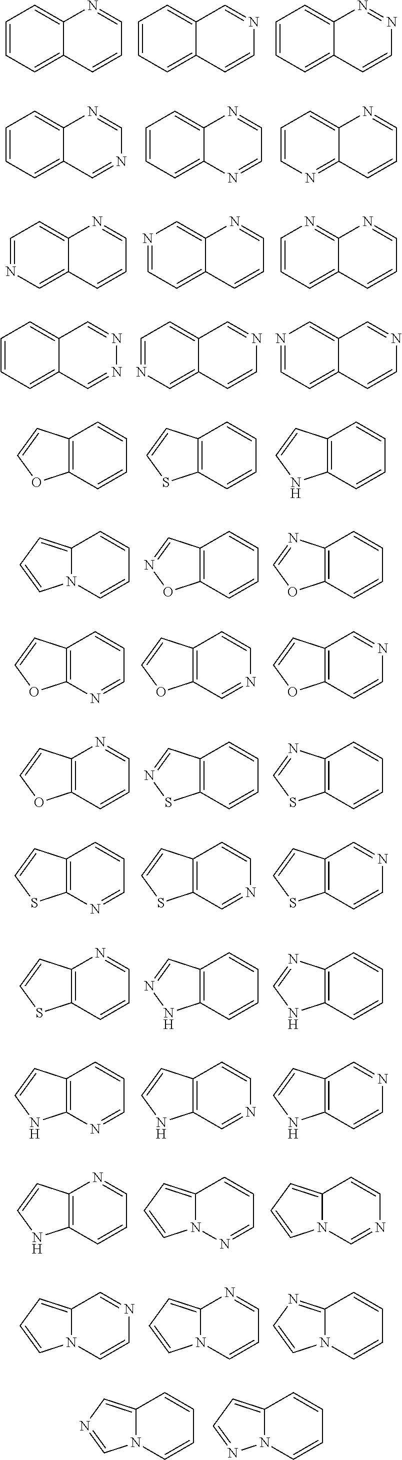

In the present specification, the “5-7 membered unsaturated heterocyclic ring” is a ring in which a monocyclic 5-7 membered saturated heterocyclic ring is partially oxidized or a ring in which an aromatic heterocyclic ring is partially reduced containing one to four atoms selected from the group consisting of a nitrogen atom, an oxygen atom, and a sulfur atom. For example, rings such as those shown below are included.

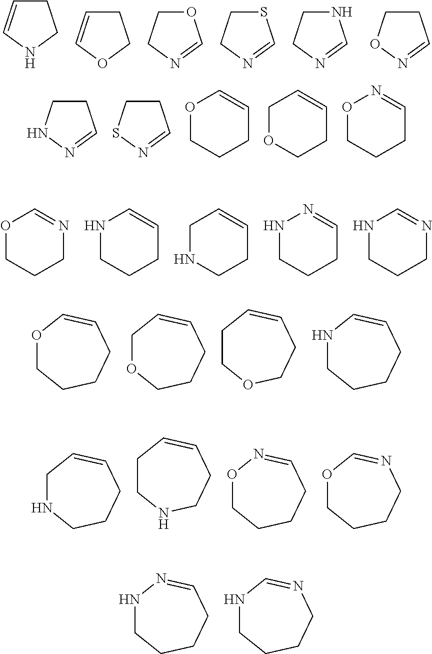

In the present specification, the “4-7 membered saturated heterocyclic ring” is a monocyclic 4-7 membered saturated heterocyclic ring containing one to four atoms selected from the group consisting of a nitrogen atom, an oxygen atom, and a sulfur atom. For example, rings such as those shown below are included.

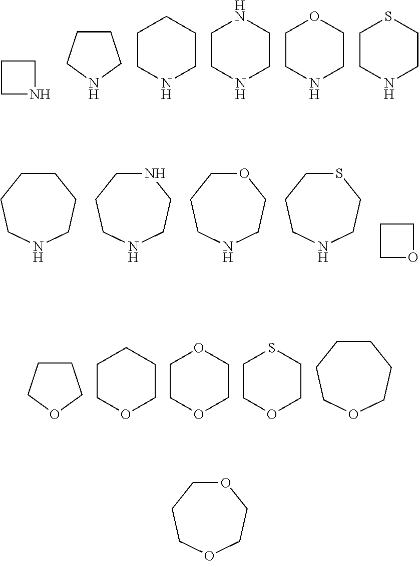

The “halogen atom” in the present specification is a fluorine atom, a chlorine atom, a bromine atom, or an iodine atom, and is preferably a fluorine atom or a chlorine atom.

The “C1-C6 alkyl group” in the present specification is a linear or branched alkyl group having one to six carbon atoms. Examples thereof include a methyl group, an ethyl group, a 1-propyl group, an isopropyl group, a 1-butyl group, a 2-butyl group, a 2-methyl-1-propyl group, a 2-methyl-2-propyl group, a 1-pentyl group, a 2-pentyl group, a 3-pentyl group, a 2-methyl-2-butyl group, a 3-methyl-2-butyl group, a 1-hexyl group, a 2-hexyl group, a 3-hexyl group, a 2-methyl-1-pentyl group, a 3-methyl-1-pentyl group, a 2-ethyl-1-butyl group, a 2,2-dimethyl-1-butyl group, and a 2,3-dimethyl-1-butyl group, and it is preferably a methyl group or an ethyl group.

The “C2-C6 alkenyl group” in the present specification is a linear or branched alkenyl group having two to six carbon atoms, and it may have one or two or more carbon-carbon double bonds. For example, it is a vinyl group, a 2-propenyl (allyl) group, a 2-butenyl group, a 2-pentenyl group, a 3-methyl-2-butenyl group, a 2-hexenyl group, or a 3-methyl-2-pentenyl group, and preferably, it is a vinyl group or an allyl group.

The “C2-C6 alkynyl group” in the present specification is a linear or branched alkynyl group having two to six carbon atoms, and it may have one or two or more carbon-carbon triple bonds. For example, it is an ethynyl group, a 1-propynyl group, a 2-propynyl group, a 1-butynyl group, a 2-butynyl group, a 1-pentynyl group, a 2-pentynyl group, or 1-hexynyl group, and it is preferably an ethynyl group or a 1-propynyl group.

The “C1-C6 alkoxy group” in the present specification is a group in which an oxygen atom is bonded to a C1-C6 alkyl group. Examples thereof include a methoxy group, an ethoxy group, a 1-propoxy group, a 2-propoxy group, a 1-butoxy group, a 2-butoxy group, a 2-methyl-1-propoxy group, a 2-methyl-2-propoxy group, a 1-pentyloxy group, a 2-pentyloxy group, a 3-pentyloxy group, a 2-methyl-2-butoxy group, a 3-methyl-2-butoxy group, a 1-hexyloxy group, a 2-hexyloxy group, a 3-hexyloxy group, a 2-methyl-1-pentyloxy group, and a 3-methyl-1-pentyloxy group. Preferably, it is a methoxy group, an ethoxy group, a 1-propoxy group, or a 2-propoxy group.

The “C3-C6 cycloalkyl group” in the present specification is a cyclic alkyl group having three to six carbon atoms, and it is preferably a cyclopropyl group, a cyclobutyl group, a cyclopentyl group, or a cyclohexyl group.

The “hydroxy C1-C6 alkyl group” in the present specification is a group in which a hydroxyl group is bonded to a C1-C6 alkyl group. For example, it is a hydroxymethyl group or a hydroxyethyl group.

The “C1-C6 alkoxy C1-C6 alkyl group” in the present specification is a group in which a C1-C6 alkoxy is bonded to a C1-C6 alkyl group. Examples thereof include a methoxymethyl group, a methoxyethyl group, an ethoxymethyl group, and an ethoxyethyl group.

The “C1-C6 haloalkyl group” in the present specification is a group in which a halogen atom is bonded to a C1-C6 alkyl group. Examples thereof include a fluoromethyl group, a difluoromethyl group, a dichloromethyl group, a dibromomethyl group, a trifluoromethyl group, a trichloromethyl group, a 2-fluoroethyl group, a 2-bromoethyl group, a 2-chloroethyl group, a 2-iodoethyl group, a 2,2-difluoroethyl group, a 2,2,2-trifluoroethyl group, a trichloroethyl group, a pentafluoroethyl group, a 3-fluoropropyl group, a 3-chloropropyl group, and a 4-fluorobutyl group. It is preferably a trifluoromethyl group.

The “C3-C6 halocycloalkyl group” in the present specification is a group in which a halogen atom is bonded to a C3-C6 cycloalkyl group, and examples thereof include a fluorocyclopropyl group, a fluorocyclobutyl group, a fluorocyclopentyl group, and a fluorocyclohexyl group.

The “C1-C6 haloalkoxy group” in the present specification is a group in which a halogen atom is bonded to a C1-C6 alkoxy group, and examples thereof include a fluoromethoxy group, a difluoromethoxy group, a dichloromethoxy group, a dibromomethoxy group, a trifluoromethoxy group, a trichloromethoxy group, a 2-fluoroethoxy group, a 2-bromoethoxy group, a 2-chloroethoxy group, a 2-iodoethoxy group, a 2,2-difluoroethoxy group, a 2,2,2-trifluoroethoxy group, a trichloroethoxy group, a pentafluoroethoxy group, a 3-fluoropropoxy group, a 3-chloropropoxy group, and a 4-fluorobutoxy group. It is preferably a trifluoromethoxy group.

The “C3-C6 cycloalkoxy group” in the present specification is a group in which a C3-C6 cycloalkyl group is bonded to an oxygen atom, and it is preferably a cyclopropyloxy group, a cyclobutyloxy group, a cyclopentyloxy group, or a cyclohexyloxy group.

The “C3-C6 halocycloalkoxy group” in the present specification is a group in which a C3-C6 halocycloalkyl group is bonded to an oxygen atom, and examples thereof include a fluorocyclopropoxy group, a fluorocyclobutoxy group, a fluorocyclopentyloxy group, and a fluorocyclohexyloxy group.

The “5-membered aromatic heterocyclic oxy group” in the present specification is a group in which a 5-membered aromatic heterocyclic ring is bonded to an oxygen atom.

The “6-membered aromatic heterocyclic oxy group” in the present specification is a group in which a 6-membered aromatic heterocyclic ring is bonded to an oxygen atom.

The “4-7 membered saturated heterocyclic oxy group” in the present specification is a group in which a 4-7 membered saturated heterocyclic ring is bonded to an oxygen atom.

The “C1-C6 alkoxycarbonyl group” in the present specification is a group in which a C1-C6 alkoxy group is bonded to a carbonyl group, and examples thereof include a methoxycarbonyl group, an ethoxycarbonyl group, and a propoxycarbonyl group.

The “C3-C6 cycloalkoxycarbonyl group” in the present specification is a group in which a C3-C6 cycloalkoxy group is bonded to a carbonyl group, and it is preferably a cyclopropyloxycarbonyl group, a cyclobutyloxycarbonyl group, a cyclopentyloxycarbonyl group, or a cyclohexyloxycarbonyl group.

The “C1-C6 alkyl carbonyl group” in the present specification is a group in which a C1-C6 alkyl group is bonded to a carbonyl group, and examples thereof include a methyl carbonyl group, an ethyl carbonyl group, or a propyl carbonyl group.

The “mono (C1-C6 alkyl) aminocarbonyl group” in the present specification is a group in which one C1-C6 alkyl group is bonded to the amino group of an aminocarbonyl group, and it is preferably a methylaminocarbonyl group, an ethylaminocarbonyl group, or a propylaminocarbonyl group.

The “di (C1-C6 alkyl) aminocarbonyl group” in the present specification is a group in which two C1-C6 alkyl groups are bonded to the amino group of an aminocarbonyl group, and it is preferably a dimethylaminocarbonyl group, a diethylaminocarbonyl group, or a dipropylaminocarbonyl group.

The “mono (C1-C6 alkyl) aminosulfonyl group” in the present specification is a group in which one C1-C6 alkyl group is bonded to the amino group of an aminosulfonyl group, and it is preferably a methylaminosulfonyl group, an ethylaminosulfonyl group, or a propylaminosulfonyl group

The “di (C1-C6 alkyl) aminosulfonyl group” in the present specification is a group in which two C1-C6 alkyl groups are bonded to the amino group of the aminosulfonyl group, and it is preferably a dimethylaminosulfonyl group, a diethylaminosulfonyl group, or a dipropylaminosulfonyl group.

The “mono (C1-C6 alkyl) amino group” in the present specification is a group in which one C1-C6 alkyl group is bonded to an amino group, and it is preferably a methylamino group, an ethylamino group, or a propylamino group.

The “di (C1-C6 alkyl) amino group” in the present specification is a group in which two C1-C6 alkyl groups are bonded to an amino group, and it is preferably a dimethylamino group, a diethylamino group, or a dipropyl amino group.

The “C1-C6 alkoxycarbonylamino group” in the present specification is a group in which a C1-C6 alkoxycarbonyl group is bonded to an amino group, and for example, it is a methoxycarbonylamino group, an ethoxycarbonylamino group, or a propoxycarbonylamino group.

The “mono (C1-C6 alkyl) aminocarbonylamino group” in the present specification is a group in which a mono (C1-C6 alkyl) aminocarbonyl group is bonded to an amino group, and it is preferably a methylaminocarbonylamino group, an ethylaminocarbonylamino group, or a propylaminocarbonylamino group.

The “di (C1-C6 alkyl) aminocarbonylamino group” in the present specification is a group in which a di (C1-C6 alkyl) aminocarbonyl group is bonded to an amino group, and it is preferably a dimethylaminocarbonylamino group, a diethylaminocarbonylamino group, or a dipropylaminocarbonylamino group.

The “5-membered aromatic heterocyclic carbonylamino group” in the present specification is a group in which a 5-membered aromatic heterocyclic carbonyl group is bonded to an amino group.

The “6-membered aromatic heterocyclic carbonylamino group” in the present specification is a group in which a 6-membered aromatic heterocyclic carbonyl group is bonded to an amino group.

The “C1-C6 alkylsulfonylamino group” in the present specification is a group in which a C1-C6 alkyl group is bonded to the sulfonyl group of a sulfonylamino group, and it is preferably a methylsulfonylamino group, an ethylsulfonylamino group, or a propylsulfonylamino group.

The “pharmaceutically acceptable salt” indicates a salt that can be used as a pharmaceutical. When the compound has an acidic group or a basic group it can be converted to a basic salt or an acidic salt by reacting with a base or an acid to form a salt thereof.

The pharmaceutically acceptable “basic salt” of the compound preferably includes an alkali metal salt such as a sodium salt, a potassium salt, and a lithium salt; an alkaline earth metal salt such as a magnesium salt and a calcium salt; organic base salts such as an N-methyl morpholine salt, a triethylamine salt, a tributylamine salt, a diisopropylethylamine salt, a dicyclohexylamine salt, an N-methylpiperidine salt, a pyridine salt, a 4-pyrrolidinopyridine salt, and a picoline salt; and an amino acid salt such as glycine salt, a lysine salt, an arginine salt, an ornithine salt, a glutamate, and an aspartate, and it is preferably an alkali metal salt.

The pharmaceutically acceptable “acidic salt” of the compound preferably includes an inorganic acid salt such as a hydrohalide such as a hydrofluoride, a hydrochloride, a hydrobromide, and a hydroiodide, a nitrate, a perchlorate, a sulfate, and a phosphate; an organic salt such as a lower alkanesulfonate such as methanesulfonate, trifluoromethanesulfonate, and ethanesulfonate, an aryl sulfonate such as a benzenesulfonates, and a p-toluene sulfonate, an acetate, a malate, a fumarate, a succinate, a citrate, an ascorbate, a tartrate, an oxalate, a maleate, and the like; and an amino acid salt such as glycine salt, a lysine salt, an arginine salt, an ornithine salt, a glutamate, and an aspartate, and it is most preferably a hydrohalide (in particular, a hydrochloride).

The compound of the present invention or the pharmaceutically acceptable salt thereof may absorb moisture, adhere to adsorbed water, or become a hydrate by leaving in the air or recrystallization. The present invention also encompasses compounds of such various hydrates, solvates, and crystalline polymorphs.

The compounds of the present invention, their pharmaceutically acceptable salts or solvates thereof, depending on the type and combination of substituents, may have various isomers such as geometric isomers such as a cis isomer and a trans isomer, tautomers, or optical isomers such as a d isomer and an 1 isomer, while the compounds include those all isomers, stereoisomers, and mixtures of these isomers and stereoisomers in any ratio unless otherwise specified. Mixtures of these isomers may be resolved by known resolution means.

The compounds of the present invention also include labels, that is, a compound in which one or more atoms of the compounds are substituted with an isotope (for example, 2H, 3H, 13C, 14C, 35S, and the like).

In addition, the present invention also encompasses a prodrug. The prodrug is a compound having a group which can be converted to an amino group, a hydroxyl group, a carboxyl group, or the like of the compound by hydrolysis or under physiological conditions, and as a group forming such a prodrug, it is a group described in Prog. Med., Vol. 5, pp. 2157 to 2161 (1985) or the like. As the prodrug, more specifically, when an amino group is present in the compound, a compound in which the amino group is acylated, alkylated, or phosphorylated (for example, it is a compound in which the amino group is eicosanoylated, alanylated, pentylaminocarbonylated, (5-methyl-2-oxo-1,3-dioxolen-4-yl) methoxycarbonylated, tetrahydrofuranylated, pyrrolidinyl methylated, pivaloyloxymethylatied, or tert-butylated, or the like) and the like are included, and when a hydroxyl group is present in the compound, a compound in which the hydroxyl group is acylated, alkylated, phosphorylated, or borated (for example, it is a compound in which the hydroxyl group is acetylated, palmitoylated, propanoylated, pivaloylated, succinylated, fumarylated, alanylated, or dimethylaminomethyl carbonylated, or the like.) and the like are included. In addition, when a carboxy group is present in the compound, a compound in which the carboxy group is esterified or amidated (for example, it is a compound in which the carboxy group is ethyl esterified, phenyl esterified, carboxymethyl esterified, dimethylaminomethyl esterified, pivaloyloxymethyl esterified, ethoxycarbonyloxyethyl esterified, amidated, or methylamidated, or the like.), and the like are included.

The compounds of the present invention may be produced by synthetic methods known in the art and as described in WO 2017/170623 and WO 2019/065928, incorporated by reference herein in their entirety.

Administration of the compounds of the present invention may be carried out by any form of oral administration by a tablet, a pill, a capsule, a granule, a powder, a solution, or the like, or by any form of parenteral administration by an injection for intra-articular, intravenous, intrathecal, intracerebroventricular, intramuscular, intranasal, or the like, a suppository, an eye drop, an eye ointment, a transdermal solution, an ointment, a transdermal patch, a transmucosal solution, a transmucosal patch, an inhalant, or the like.

As a solid composition for oral administration, a tablet, a powder, a granule, and the like are used. Such a solid composition is composed of one or more active ingredients and at least one inert excipient such as lactose, mannitol, glucose, hydroxypropyl cellulose, microcrystalline cellulose, starch, polyvinyl pyrrolidone, magnesium metasilicate aluminate, and/or the like. The solid composition may contain, according to a conventional method, one or more of an inert additive such as a lubricant such as magnesium stearate, a disintegrant such as sodium carboxymethyl starch, a stabilizer, and a solubilizer. The tablet or pill may be coated with a sugar coating or a film of a substance soluble in the stomach or intestine, if necessary.

As a liquid composition for oral administration, a pharmaceutically acceptable emulsion, solution, suspension, syrup, elixir, or the like is used. To such a liquid composition, it is possible to add a generally used inert diluent such as purified water or ethanol. The liquid composition may contain, in addition to an inert diluent, one or more of a solubilizer, an adjuvant such as a wetting agent, a sweetening agent, a flavoring agent, a fragrance, and a preservative.

As an injection for parenteral administration, a sterile aqueous or non-aqueous solution, a suspension or an emulsion, and the like are used. The aqueous solvent includes, for example, distilled water for injection, physiological saline, and the like. The non-aqueous solvents include, for example, propylene glycol, polyethylene glycol, and vegetable oil such as olive oil, alcohols such as ethanol, Polysorbate 80, and the like. Such an injection composition may further contain a one or more of a tonicity agent, a preservative, a wetting agent, an emulsion, a dispersing agent, a stabilizer, or a solubilizer. These injection compositions can be sterilized by, for example, filtration through a bacteria retention filter, application of a bactericide, or irradiation. In addition, these injection compositions may be used by producing a sterile solid composition and dissolved or suspended in sterile water or a sterile solvent for injection prior to use.

As an external preparation, an ointment, a plaster, a cream, a jelly, a cataplasm, a spray, a lotion, an eye drop, an eye ointment, and the like are used. These external preparations include generally used ointment bases, lotion bases, aqueous or non-aqueous solutions, suspensions, emulsions, and the like. For example, as an ointment or lotion base, polyethylene glycol, propylene glycol, white petrolatum, bleached beeswax, polyoxyethylene hydrogenated castor oil, glycerin monostearate, stearyl alcohol, cetyl alcohol, lauromacrogol, sorbitan sesquioleate, and the like are used.

A transmucosal agent such as an inhalant and a transnasal agent are used in solid, liquid, or semisolid form, and it may be produced according to a conventionally known method. For example, a known excipient, and furthermore, one or more of a pH adjuster, a preservative, a surfactant, a lubricant, a stabilizer, a thickener, and the like may be added as appropriate. With these transmucosal agents, devices appropriate for inhalation or insufflation may be used as the method of administration. For example, the compound may be administered alone or as a powder of a formulated mixture, or as a solution or suspension in combination with a pharmaceutically acceptable carrier, using known devices and nebulizers, such as metered dose inhalation devices. A dry powder inhaler or the like may be for single or multiple administration, and a dry powder or powder containing capsule may be also used. Alternatively, an appropriate ejector may be used. For example, it may be in the form of a pressurized aerosol spray or the like using a suitable gas such as chlorofluoroalkane, hydrofluoroalkane, or carbon dioxide.

In the case of normal oral administration, the appropriate daily dose is about 0.001 to 100 mg/kg, preferably 0.1 to 30 mg/kg, and more preferably 0.1 to 10 mg/kg of body weight. In some embodiments, the appropriate daily dose is about 0.1 mg to about 500 mg, e.g., about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 mg. This is administered in one dose or separated into two or more doses. When administered intravenously, the appropriate daily dose is about 0.0001 to 10 mg/kg of body weight, which is administered once or separated into several times a day. In addition, as a transmucosal agent, about 0.001 to 100 mg/kg of body weight is administered once or separated into several times a day. The dose is appropriately determined depending on the individual case in consideration of symptoms, age, sex, and the like.

In the methods of the present invention, the compound may be administered in combination with various therapeutic agents or preventive agents for diseases that are considered to exhibit the efficacy thereof. The combination may be administered simultaneously, separately, concurrently, and continuously or at desired time intervals. The co-administered agents may be blended or formulated separately. The therapeutic agent may be, for example, one that treats conditions causing memory, cognition, or learning issues, including benzodiazepines, anticholinergics, antihistamines, opioids, proton pump inhibitors, antidepressants, hypertension treatments, sleep apnea treatments, treatments to boost neurotransmitter levels (e.g., donepezil, galantamine, rivastigmine), memantine, aducanumab, and suvorexant.

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a compound as described herein. As a further option, the subject can be a laboratory animal and/or an animal model of disease. Preferably, the subject is a human.

Having described the present invention, the same will be explained in greater detail in the following examples, which are included herein for illustration purposes only, and which are not intended to be limiting to the invention.

### Example 1

### Bioactivity of Compound 1

Compound 1 (SP-624; DS-7830a; (2S,6′R)-7-Chloro-2′,4-dimethoxy-6′-methyl-6-(5-methyl-1,3,4-oxadiazol-2-yl)-3H-spiro[1-benzofuran-2,1′-cyclohex[2]ene]-3,4′-dione) was tested in multiple models of cognition.

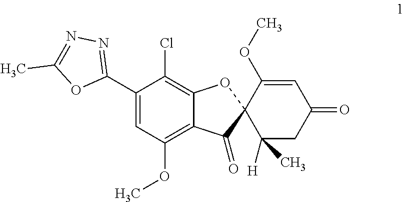 [SMILES: COC1=CC(=O)C[C@@H](C)[C@]12Oc1c(Cl)c(-c3nnc(C)o3)cc(OC)c1C2=O]

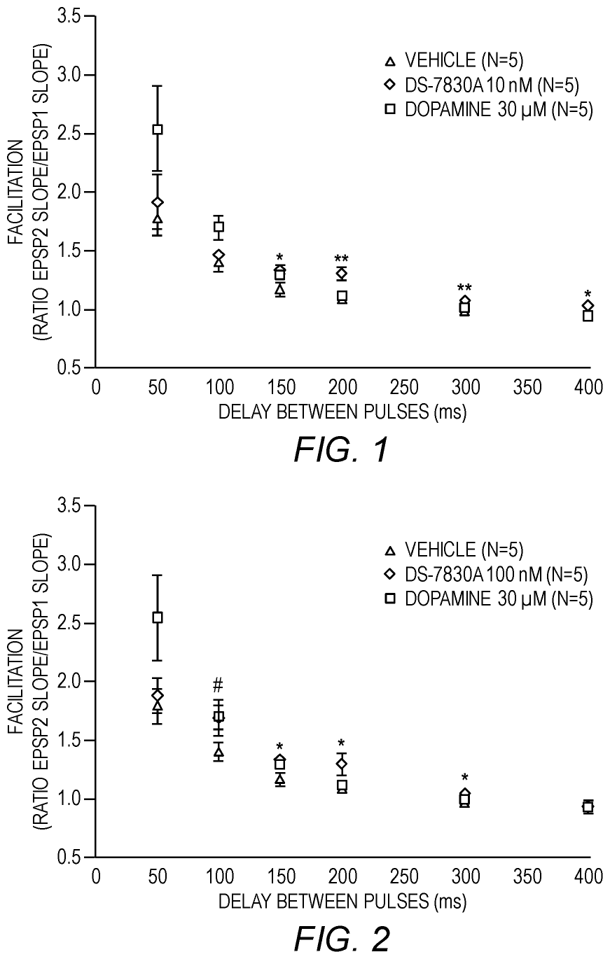

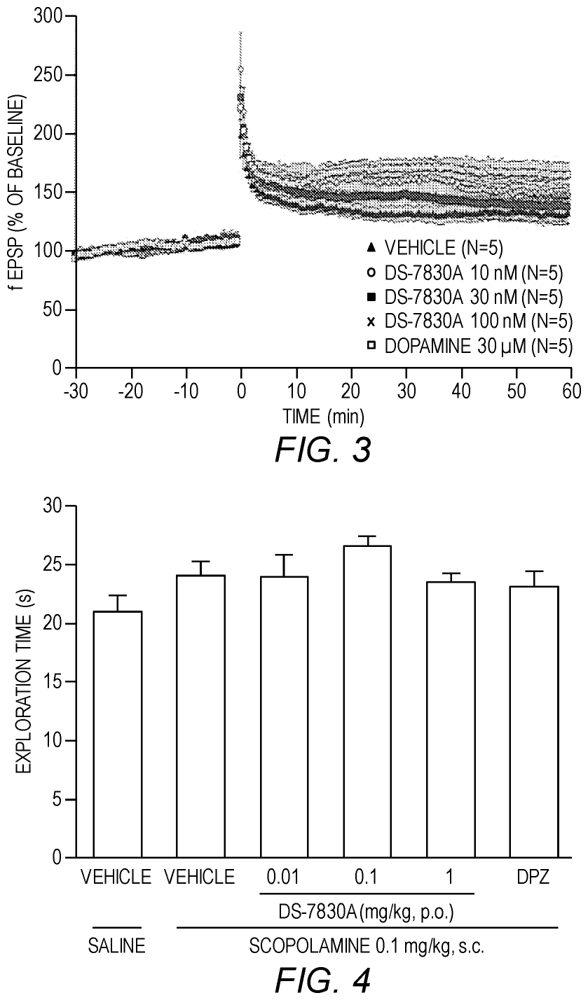

Ex vivo electrophysiology experiments conducted in Sprague Dawley rat hippocampal slices demonstrated that compound 1 acts presynaptically to increase the frequency of miniature excitatory post-synaptic potentials (mEPSPs) in the hippocampus and enhanced short-term plasticity (FIGS. 1-2) and tended to increase short-lasting long-term potentiation (FIG. 3), suggesting the facilitation of neurotransmitter release in hippocampus. The effect of compound 1 to enhance mEPSPs may be dependent on the influx of extracellular calcium. The results suggested that the influx of extracellular calcium primarily involves P/Q type and N-type calcium channels, but not L-type calcium channels or protein kinase A. These findings indicate enhancement of neuronal transmission in hippocampus, a brain region involved in memory and cognitive function.

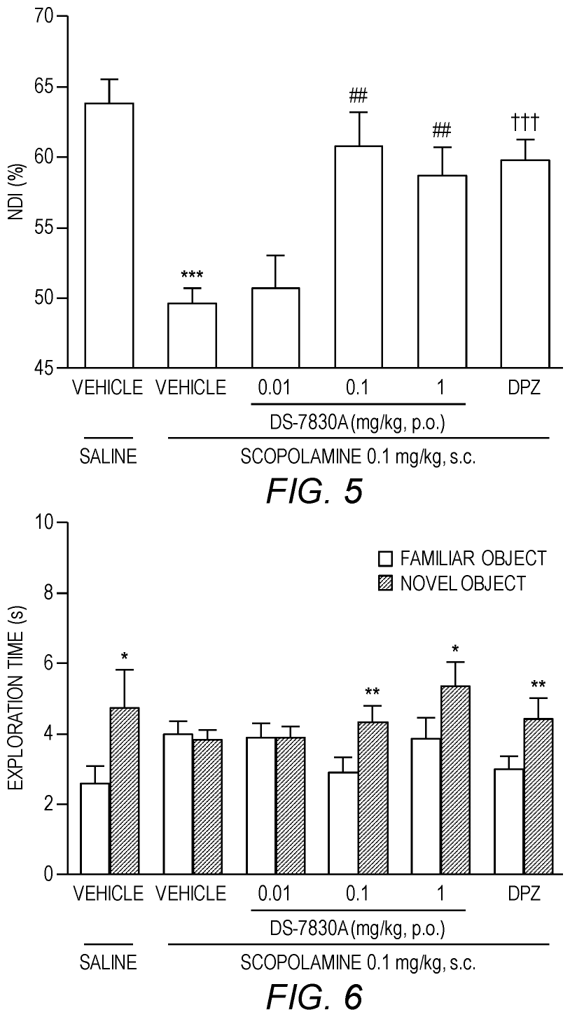

A novel object recognition test (which evaluates visual learning and memory) was conducted to investigate the effect of compound 1 on scopolamine-induced cognitive impairment in Long-Evans rats. Donepezil was used as a positive control. Scopolamine at 0.1 mg/kg SC reduced the novelty discrimination index (NDI) a measure of cognitive function (FIGS. 4-6). Compound 1 at 0.1 and 1 mg/kg PO significantly improved the NDI reduced by scopolamine. This suggests that compound 1 improves scopolamine-induced cognitive impairment.

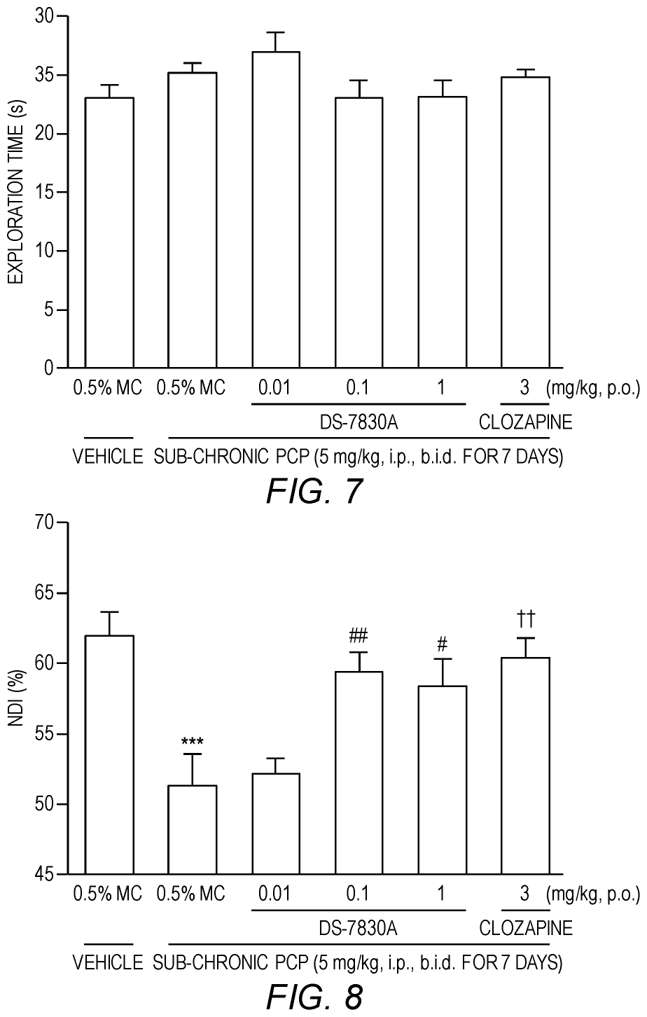

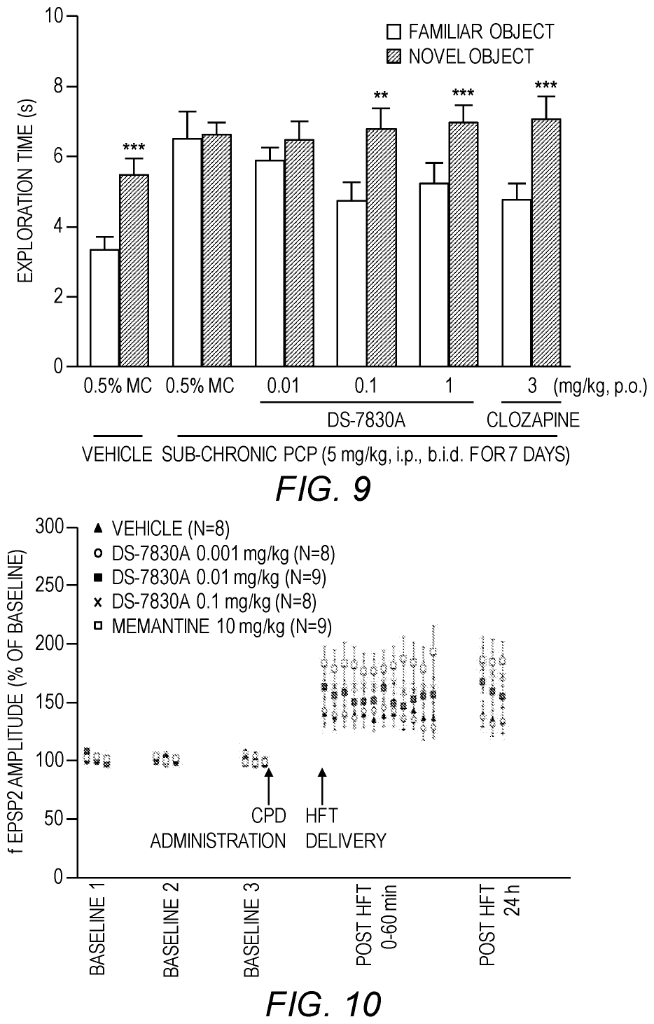

A novel object recognition test was conducted to investigate the effect of SP-624 on subchronic PCP-induced cognitive impairment in Wistar rats. Clozapine was used as a positive control. Sub-chronic PCP at 5 mg/kg twice daily for 7 days IP significantly decreased the NDI compared with sub-chronic vehicle treatment (FIGS. 7-9). Treatment with compound 1 at 0.1 and 1 mg/kg, but not 0.01 mg/kg, significantly attenuated the sub-chronic PCP-induced cognitive deficits in the NDI. This result suggests that compound 1 ameliorates cognitive deficits involved in NMDA receptor hypofunction.

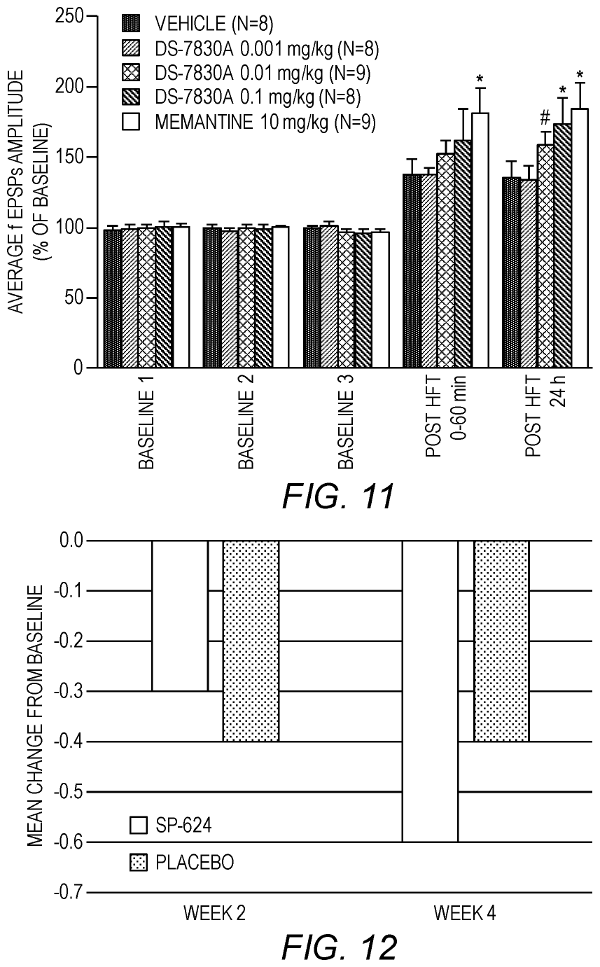

A freely behaving rodent model of long-term potentiation (LTP), a frequently studied form of synaptic plasticity, was employed. LTP is a persistent increase in the strength of synaptic connections between neurons, and is a cellular mechanism widely considered to underlie the processes of learning and memory. At 0.01 and 0.1 mg/kg IV compound 1 increased LTP compared to vehicle controls (FIGS. 10-11). The results indicate that compound 1 may positively effect cognition.

### Example 2

### Clinical Trial of Compound 1

A Phase 2, multicenter, double-blind, randomized, placebo-controlled study of the safety and efficacy of SP-624 in the treatment of adult subjects with major depressive disorder (MDD) as defined by the Diagnostic and Statistical Manual of Mental Disorders, Fifth Edition (DSM-5) was carried out. Following the successful completion of a Screening Phase, subjects were randomized to one of two treatment groups in a 1:1 ratio and received Compound 1 (SP-624) or placebo over a treatment period of four weeks (Treatment Group #1: SP-624 20 mg/day; Treatment Group #2: Placebo).

Prior to initiating the 4-week treatment period, subjects completed a Screening/Baseline period of up to 28 days, during which time all screening assessments were performed, and any current depression medications discontinued. All screening assessments were completed before discontinuing any current depression medications. Subjects returned for a Baseline Visit to complete efficacy and safety assessments. During the treatment period, subjects returned to the investigative site to complete efficacy and safety assessments at the end of Weeks 1, 2, 3, and 4. After the final dose of study drug, subjects completed a follow-up period of two weeks and returned at the end of Weeks 5 and 6. Subjects may be treated with anti-depressant medications according to physician recommendations after completion of the Week 5 Visit.

Each dose of Compound 1 was supplied as two capsules, each containing 10 mg of active pharmaceutical ingredient (API). Matching placebo capsules identical in shape and color to the active capsules were used.

The primary efficacy endpoint was Change from Baseline to Week 4 in Montgomery-Asberg Depression Rating Scale (MADRS) total score.

Secondary efficacy endpoints included:
Change from Baseline to Weeks 1, 2, and 3 in MADRS total score,
Change from Baseline to Weeks 1, 2, 3, and 4 in Clinical Global Impression—Severity (CGI-S) total score,
Change from Baseline to Week 5 and change from Week 4 to Week 5 in MADRS total score and CGI-S total score,
Change from Baseline to Week 2 and Week 4 in the 17-item Hamilton Depression Rating Scale (HAM D-17) total score,
Change from Baseline to Week 2 and Week 4 in the Sheehan Disability Scale (SDS),
Change from Baseline to Week 2 and Week 4 in the Quick Inventory of Depressive Symptomology— Self Report (QIDS-SR), and
Change from Baseline to Week 2 and Week 4 in the Quality of Life Enjoyment and Satisfaction Questionnaire— Short Form (Q-LES-Q-SF).

The demographics of the study population is shown in Table 1.

TABLE 1
Demographics, Safety Population

| | SP-624 | Placebo | Overall |
|---|---|---|---|
| Summary | (N = 161) | (N = 156) | (N = 317) |
| Age (Years) | | | |
| n | 161 | 156 | 317 |
| Mean (SD) | 41.5 (13.95) | 43.9 (14.11) | 42.7 (14.06) |
| Median | 42.0 | 44.0 | 43.0 |
| Min-Max | 18.0-65.0 | 18.0-65.0 | 18.0-65.0 |
| Gender, n (%) | | | |
| Female | 101 (62.7%) | 110 (70.5%) | 211 (66.6%) |
| Male | 60 (37.3%) | 46 (29.5%) | 106 (33.4%) |
| Race | | | |
| American Indian or | 2 (1.2%) | 2 (1.3%) | 4 (1.3%) |
| Alaska native | | | |
| Asian | 8 (5.0%) | 12 (7.7%) | 20 (6.3%) |
| Black or African | 41 (25.5%) | 34 (21.8%) | 75 (23.7%) |
| American | | | |
| Native Hawaiian or | 2 (1.2%) | 2 (1.3%) | 4 (1.3%) |
| other Pacific Islander | | | |
| White | 113 (70.2%) | 111 (71.2%) | 224 (70.7%) |
| Ethnicity, n (%) | | | |
| Hispanic or Latino | 16 (9.9%) | 21 (13.5%) | 37 (11.7%) |
| Not Hispanic or Latino | 142 (88.2%) | 135 (86.5%) | 277 (87.4%) |
| Nor reported | 1 (0.6%) | 0 (0.0%) | 1 (0.3%) |
| Unknown | 2 (1.2%) | 0 (0.0%) | 2 (0.6%) |

The study included several measurements related to cognitive function. The first is the QIDS-SR) Item #10 Concentration/Decision Making. The answer scale is: 0—There is no change in my usual capacity to concentrate or make decisions; 1—I occasionally feel indecisive or find that my attention wanders; 2—Most of the time, I struggle to focus my attention or to make decisions; 3—I cannot concentrate well enough to read or cannot make even minor decisions. Results are shown in FIG. 12 and Table 2. The data show that by week 4 of treatment there was a greater improvement in score in the subjects administered Compound 1.

TABLE 2
QIDS-SR - Item # 10 (Concentration/Decision
Making) -Mean Change from Baseline (mITT, Females)

| | | | Change from Baseline | |
|---|---|---|---|---|
| | SP-624 | Placebo | SP-624 | Placebo |
| | (N = 98) | (N = 107) | (N = 98) | (N = 107) |
| QIDS Q10 | | | | |
| Concentration/ | | | | |
| Decision | | | | |
| Making at Baseline | | | | |
| n | 98 | 107 | | |
| Mean (SD) | 1.7 (0.66) | 1.9 (0.72) | | |
| Median | 2.0 | 2.0 | | |
| Min | 0 | 0 | | |
| Max | 3 | 3 | | |
| QIDS Q10 | | | | |
| Concentration/ | | | | |
| Decision | | | | |
| Making at Week 2 | | | | |
| n | 86 | 92 | 86 | 92 |
| Mean (SD) | 1.5 (0.82) | 1.6 (0.87) | −0.3 (0.71) | −0.4 (0.85) |
| Median | 2.0 | 2.0 | 0.0 | 0.0 |
| Min | 0 | 0 | −3 | −3 |
| Max | 3 | 3 | 1 | 1 |
| QIDS Q10 | | | | |
| Concentration/ | | | | |
| Decision | | | | |
| Making at Week 4 | | | | |
| n | 83 | 85 | 83 | 85 |
| Mean (SD) | 1.2 (0.86) | 1.5 (0.88) | −0.6 (0.80) | −0.4 (0.89) |
| Median | 1.0 | 2.0 | 0.0 | 0.0 |
| Min | 0 | 0 | −3 | −3 |
| Max | 3 | 3 | 1 | 1 |

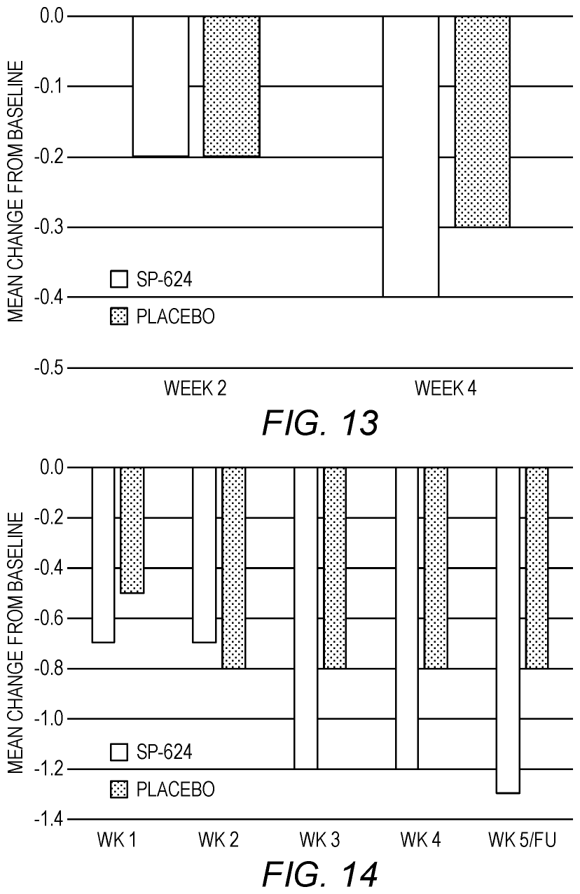

The second cognitive function measurement is HAM-D Item #17 Retardation (slowness of thought and speech, impaired ability to concentrate, decreased motor activity). The answer scale is: 0—Normal speech and thought; 1—Slight retardation at interview (mild psychomotor retardation); 2—Obvious retardation at interview (moderate, some difficulty with interview, noticeable pauses and slowness of thought); 3—Interview difficult (severe psychomotor retardation, very long pauses); 4—Complete stupor (extreme retardation, interview barely possible). The results are shown in FIG. 13 and Table 3. The data show that by week 4 of treatment there was a greater improvement in score in the subjects administered Compound 1.

TABLE 3
HAM-D - Item # 17 (Retardation) -
Mean Change from Baseline (mITT, Females)

| | | | Change From Baseline | |
|---|---|---|---|---|
| | SP-624 | Placebo | SP-624 | Placebo |
| | (N = 98) | (N = 107) | (N = 98) | (N = 107) |
| HAM-D Q17 | | | | |
| Retardation | | | | |
| at Baseline | | | | |
| n | 98 | 107 | | |
| Mean (SD) | 0.8 (0.79) | 0.9 (0.79) | | |
| Median | 1 | 1 | | |
| Min | 0 | 0 | | |
| Max | 2 | 2 | | |
| HAM-D Q17 | | | | |
| Retardation | | | | |
| at Week 2 | | | | |
| n | 86 | 92 | 86 | 92 |
| Mean (SD) | 0.6 (0.71) | 0.7 (0.77) | −0.2 (0.79) | −0.2 (0.80) |
| Median | 0 | 1 | 0 | 0 |
| Min | 0 | 0 | −2 | −2 |
| Max | 2 | 2 | 2 | 2 |
| HAM-D Q17 | | | | |
| Retardation | | | | |
| at Week 4 | | | | |
| n | 84 | 86 | 84 | 86 |
| Mean (SD) | 0.4 (0.55) | 0.6 (0.67) | −0.4 (0.82) | −0.3 (0.75) |
| Median | 0 | 0 | 0 | 0 |
| Min | 0 | 0 | −2 | −2 |
| Max | 2 | 2 | 1 | 2 |

The third cognitive function measurement is MADRS Item #6 Concentration Difficulties (representing difficulties in collecting one's thoughts mounting to incapacitating lack of concentration, rated according to to intensity, frequency, and degree of incapacity produced). The answer scale is: 0—No difficulties in concentration; 1—blank; 2—Occasional difficulties in collecting one's thoughts; 3—blank; 4—Difficulties in concentrating and sustaining thought which reduces ability to read or hold a conversation; 5—blank; 6—Unable to read or converse without great difficulty. The results are shown in FIG. 14 and Table 4. The data show that at all measurement times through week 5 there was a greater improvement in score in the subjects administered Compound 1.

TABLE 4
MADRS - Item # 6 (Concentration Difficulty) - Mean Change from Baseline (mITT, Females)

| | | | Change from Baseline | | | |
|---|---|---|---|---|---|---|
| | | | | | LSM | |
| | SP-624 | Placebo | SP-624 | Placebo | Difference¹ | |
| | (N = 98) | (N = 107) | (N = 98) | (N = 107) | (95% CI) | P-value¹ |
| MADRS Q6 Concentration | | | | | | |
| Difficulty at Baseline | | | | | | |
| n | 98 | 107 | | | | |
| Mean (SD) | 3.8 (1.01) | 3.7 (1.04) | | | | |
| Median | 4 | 4 | | | | |
| Min, Max | 0, 6 | 0, 6 | | | | |
| MADRS Q6 Concentration | | | | | | |
| Difficulty at Week 1 | | | | | | |
| n | 95 | 105 | 95 | 105 | −0.2 (−0.5, 0.2) | 0.339 |
| Mean (SD) | 3.0 (1.48) | 3.2 (1.34) | −0.7 (1.27) | −0.5 (1.31) | | |
| Median | 4 | 4 | 0 | 0 | | |
| Min, Max | 0, 6 | 0, 5 | −5, 1 | −4, 3 | | |
| LSM Estimate (SE) | | | −0.7 (0.13) | −0.5 (0.12) | | |
| MADRS Q6 Concentration | | | | | | |
| Difficulty at Week 2 | | | | | | |
| n | 86 | 92 | 86 | 92 | 0.1 (−0.3, 0.4) | 0.753 |
| Mean (SD) | 3.0 (1.37) | 2.9 (1.47) | −0.7 (1.27) | −0.8 (1.50) | | |
| Median | 3 | 4 | 0 | 0 | | |
| Min, Max | 0, 5 | 0, 4 | −4, 3 | −4, 4 | | |
| LSM Estimate (SE) | | | −0.7 (0.14) | −0.8 (0.13) | | |
| MADRS Q6 Concentration | | | | | | |
| Difficulty at Week 3 | | | | | | |
| n | 89 | 90 | 89 | 90 | −0.3 (−0.7, 0.2) | 0.214 |
| Mean (SD) | 2.6 (1.58) | 2.9 (1.56) | −1.2 (1.40) | −0.8 (1.60) | | |
| Median | 3 | 4 | −1 | 0 | | |
| Min, Max | 0, 4 | 0, 5 | −5, 0 | −4, 4 | | |
| LSM Estimate (SE) | | | −1.1 (0.15) | −0.8 (0.15) | | |
| MADRS Q6 Concentration | | | | | | |
| Difficulty at Week 4 | | | | | | |
| n | 84 | 86 | 84 | 86 | −0.3 (−0.8, 0.1) | 0.144 |
| Mean (SD) | 2.5 (1.67) | 2.9 (1.52) | −1.2 (1.51) | −0.8 (1.53) | | |
| Median | 3 | 4 | −1 | 0 | | |
| Min, Max | 0, 5 | 0, 5 | −4, 1 | −4, 4 | | |
| LSM Estimate (SE) | | | −1.1 (0.16) | −0.8 (0.15) | | |
| MADRS Q6 Concentration | | | | | | |
| Difficulty at Week 5* | | | | | | |
| n | 80 | 79 | 80 | 79 | −0.6 (−1.1, −0.2) | 0.007 |
| Mean (SD) | 2.4 (1.63) | 2.9 (1.64) | −1.3 (1.51) | −0.8 (1.67) | | |
| Median | 3 | 4 | −1 | 0 | | |
| Min, Max | 0, 5 | 0, 6 | −5, 1 | −4, 4 | | |
| LSM Estimate (SE) | | | −1.4 (0.16) | −0.8 (0.16) | | |

¹P-value, difference in Least Square Means estimate and 95% CI based on Mixed Model for Repeated Measures.
*Week 5 assessments were completed 1-week after the last dose of study drug. Week 5 summary includes subjects who received the complete four weeks of study drug, and returned for Week 5 assessments.

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A method of improving memory function, cognition function, and/or learning function in a subject in need thereof, comprising administering to the subject a therapeutically effective amount of the compound of Formula 1 or a pharmaceutically acceptable salt thereof: 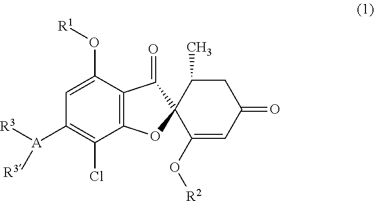
wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH=, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
substituent group Y is a C1-C6 alkyl group, a C1-C6 alkoxy group, a halogen atom, or a hydroxy group,
thereby improving memory function, cognition function, and/or learning function in the subject.

2. A method of delaying or slowing a decrease in memory function, cognition function, and/or learning function in a subject in need thereof, comprising administering to the subject a therapeutically effective amount of a compound of Formula 1 or a pharmaceutically acceptable salt thereof: 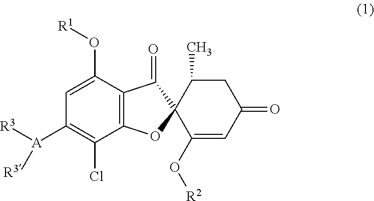
wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH=, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
substituent group Y is a C1-C6 alkyl group, a C1-C6 alkoxy group, a halogen atom, or a hydroxy group,
thereby delaying or slowing the decrease in memory function, cognition function, and/or learning function in the subject.

3. The method of claim 1, wherein the subject does not have a neurological disorder or brain injury.

4. The method of claim 1, wherein the subject has normal memory function, cognition function, and/or learning function relative to the general population.

5. The method of claim 1, wherein the subject has reduced memory function, cognition function, and/or learning function relative to the general population.

6. The method of claim 5, wherein the reduced memory function, cognition function, and/or learning function is due to aging.

7. The method of claim 1, wherein the 5-membered aromatic heterocyclic ring or the 5-membered aromatic heterocyclic group in A, R³, or R^{3′} is any one selected from the group:

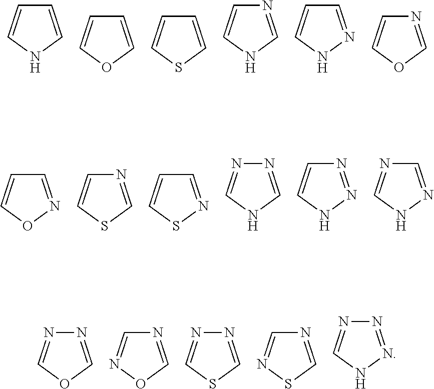

8. The method of claim 1, wherein the 6-membered aromatic heterocyclic ring or the 6-membered aromatic heterocyclic group in A, R³, or R^{3′} is any one selected from the group:

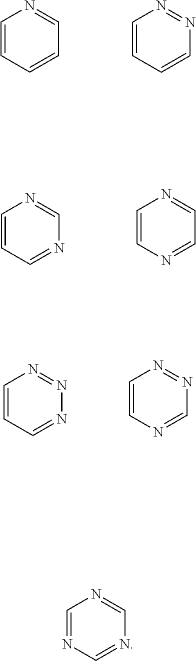

9. The method of claim 1, wherein the 8-10 membered condensed aromatic heterocyclic ring or the 8-10 membered condensed aromatic heterocyclic group in A, R³, or R^{3′} is any one selected from the group:

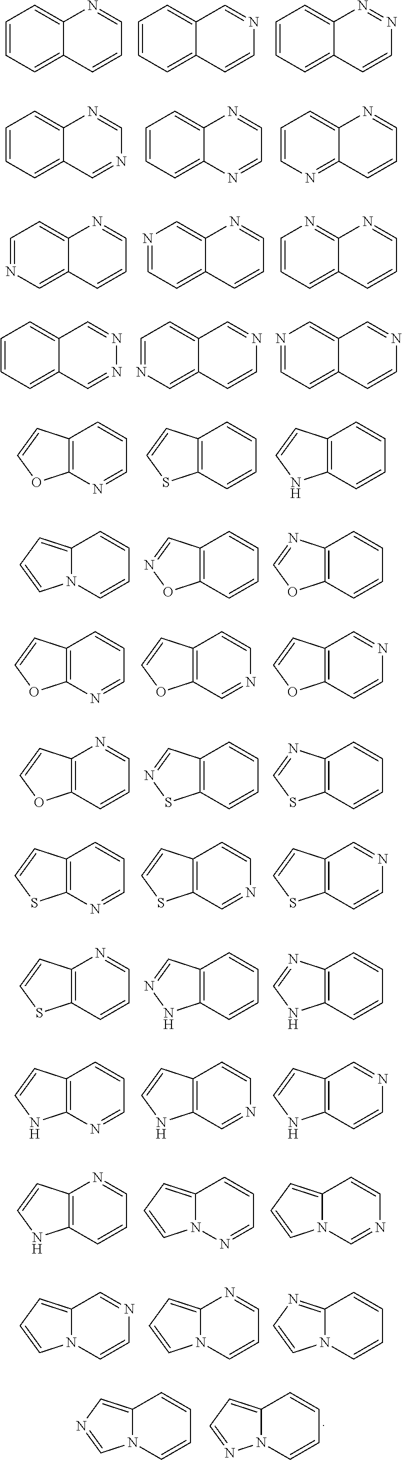

10. The method of claim 1, wherein the 5-7 membered unsaturated heterocyclic ring or 5-7 membered unsaturated heterocyclic group in A, R³, or R^{3′} is any one selected from the group:

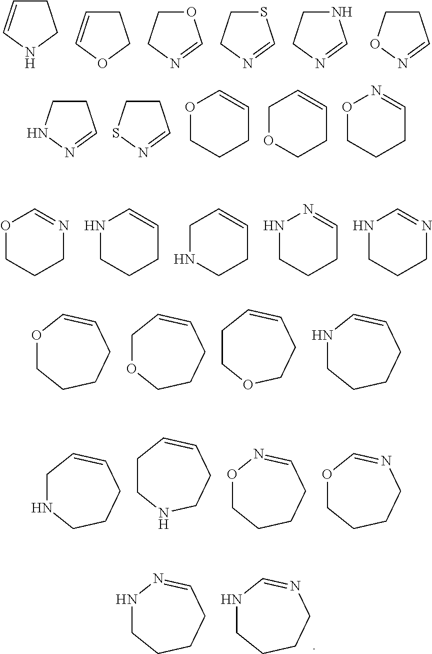

11. The method of claim 1, wherein the 4-7 membered saturated heterocyclic ring or the 4-7 membered saturated heterocyclic group in A, R¹, R², or R³ is any one selected from the group:

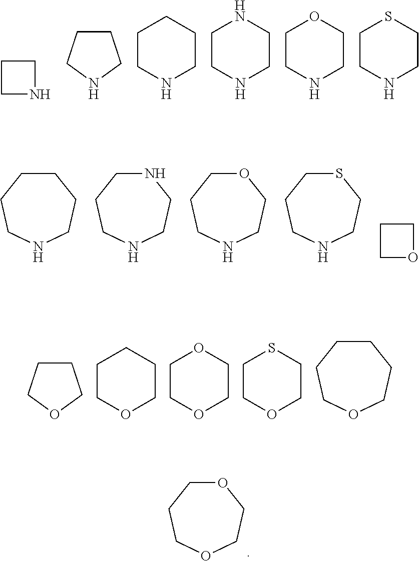

12. The method of claim 1, wherein the compound of Formula 1 is any compound selected from the following group:
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl]spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5]dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione; or
(2S,5′R)-7-chloro-3′,4-dimethoxy-5′-methyl-6-(3-pyridyl) spiro[benzofuran-2,4′-cyclohex-2-ene]-1′,3-dione.

13. The method of claim 1, wherein the compound of Formula 1 is a compound of Formula 1′ or a pharmacologically acceptable salt thereof: 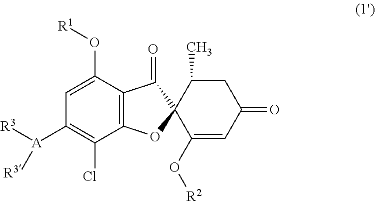
wherein:
R¹ is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, or a single bond, wherein when it is a single bond, one or the other of R³ and R^{3′} is not present,
R³ and R^{3′} are each independently a hydrogen atom, a halogen atom, a cyano group, a hydroxy group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X,
or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from the substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group, a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, and
substituent group Y is a C1-C6 alkyl group, a C1-C6 alkoxy group, a halogen atom, or a hydroxy group.

14. The method of claim 13, wherein R¹ is a methyl group, an ethyl group, or a hydroxyethyl group.

15. The method of claim 13, wherein R² is a methyl group.

16. The method of claim 13, wherein the 5-membered aromatic heterocyclic ring or the 5-membered aromatic heterocyclic group in A, R³, or R^{3′} is any one selected from the group:

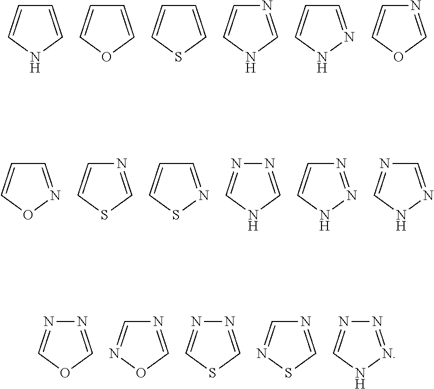

17. The method of claim 13, wherein the 6-membered aromatic heterocyclic ring or the 6-membered aromatic heterocyclic group in A, R³, or R^{3′} is any one selected from the group:

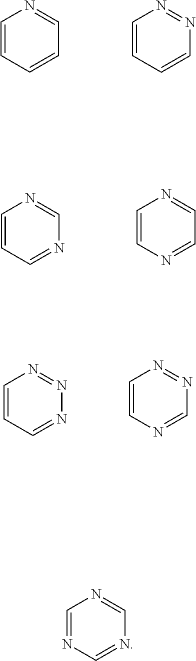

18. The method of claim 13, wherein the 5-7 membered unsaturated heterocyclic ring or the 5-7 membered unsaturated heterocyclic group in A, R³, or R^{3′} is any one selected from the group:

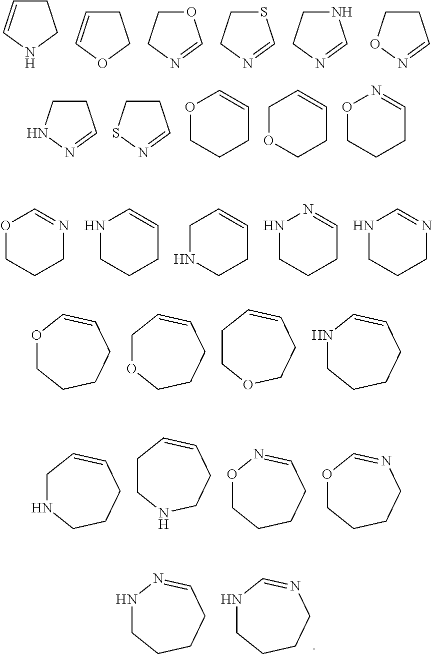

19. The method of claim 13, wherein the 4-7 membered saturated heterocyclic ring or the 4-7 membered saturated heterocyclic group in A, R¹, R², or R³ is any one selected from the group:

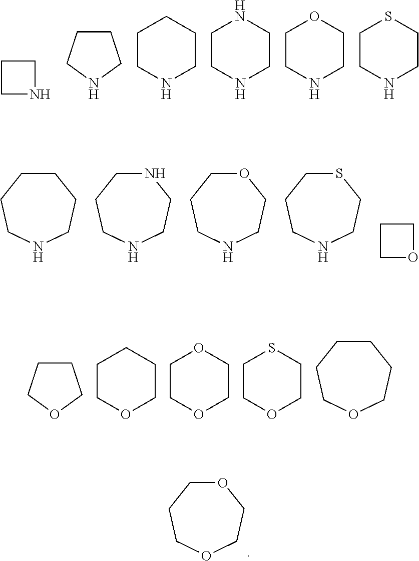

20. The method of claim 13, wherein A is a 5-membered aromatic heterocyclic ring, R³ is a methyl group, an ethyl group, a hydroxy C1-C3 alkyl group, or a methoxy C1-C3 alkyl group, and R^{3′} is a hydrogen atom.

21. The method of claim 13, wherein A is any ring selected from the following group, and in the case of two binding groups, R^{3′} is not present: 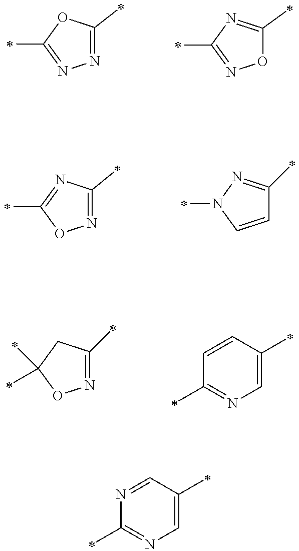
wherein * indicates a binding group.

22. The method of claim 13, wherein the compound of Formula 1 is a compound of a Formula 1″ or a pharmacologically acceptable salt thereof: 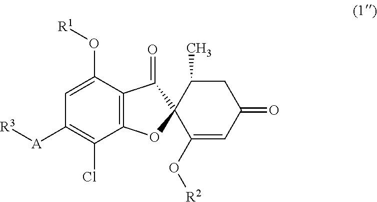
wherein R¹ is a methyl group or an ethyl group;
R² is a methyl group;
A is any ring selected from the following group: 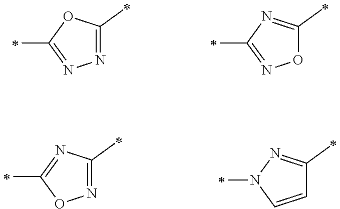
* indicates a binding group; and
R³ is a methyl group or an ethyl group.

23. The method of claim 13, wherein the compound of Formula 1′ is any compound selected from the following group:
(2S,5′R)-7-chloro-6-(2-hydroxyethoxy)-3′,4-dimethoxy-5′-methyl-spiro[benzofuran-2,4′-cyclohex-2-ene]-1′,3-dione;
(2S,5′R)-7-chloro-3′,4-dimethoxy-6-(2-methoxyethoxy)-5′-methyl-spiro[benzofuran-2,4′-cyclohex-2-ene]-1′,3-dione;
(2S,5′R)-7-chloro-3′,4-dimethoxy-5′-methyl-6-(1-methylpyrazol-3-yl) spiro[benzofuran-2,4′-cyclohex-2-ene]-1′,3-dione;
(2S,5′R)-7-chloro-6-(1-ethylpyrazol-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-(3-methyl-1,2,4-oxadiazol-5-yl) spiro[benzofuran-2,4′-cyclohex-2-ene]-1′,3-dione;
(2S,5′R)-7-chloro-3′,4-dimethoxy-5′-methyl-6-(5-methyl)-1,2,4-oxadiazol-3-yl) spiro[benzofuran-2,4′-cyclohex-2-ene]-1′,3-dione;
(2S,5′R)-7-chloro-6-[5-(1-hydroxy-1-methyl-ethyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-6-[5-[(1S)-1-hydroxyethyl]-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-6-[5-[(1R)-1-hydroxyethyl]-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-4-ethoxy-6-[5-(1-hydroxy-1-methyl-ethyl)-1,3,4-oxadiazol-2-yl]-3′-methoxy-5′-methyl-spiro[benzofuran-2,4′-cyclohex-2-ene]-1′,3-dione;
(2S,5′R)-7-chloro-4-ethoxy-6-[5-[(1S)-1-hydroxyethyl]-1,3,4-oxadiazol-2-yl]-3′-methoxy-5′-methyl-spiro[benzofuran-2,4′-cyclohex-2-ene]-1′,3-dione;
(2S,5′R)-7-chloro-6-[3-(1-hydroxyethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(2-hydroxyethoxy)-3′-methoxy-5′-methyl-6-(3-methyl-1,2,4-oxadiazol-5-yl) spiro[benzofuran-2,4′-cyclohex-2-ene]-1′,3-dione;
(2S,5′R)-7-chloro-4-(2-hydroxyethoxy)-3′-methoxy-5′-methyl-6-(5-methyl-1,2,4-oxadiazol-3-yl) spiro[benzofuran-2,4′-cyclohex-2-ene]-1′,3-dione;
(2S,5′R)-7-chloro-6-(5-ethyl-1,3,4-oxadiazol-2-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro[benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl]spiro[benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro[benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro[benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro[benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro[benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(1H-pyrazol-5-yl) spiro[benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro[benzofuran-2,4′-cyclohex-2-ene]-1′,3-dione;
(2S,5′R)-7-chloro-6-(1,8-dioxa-2-azaspiro[4.5]dec-2-en-3-yl)-3′,4-dimethoxy-5′-methyl-spiro[benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5]dec-2-en-3-yl) spiro[benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro[benzofuran-2,4′-cyclohex-2-ene]-1′,3-dione;
(2S,5′R)-7-chloro-3′,4-dimethoxy-6-(6-methoxy-3-pyridyl)-5′-methyl-spiro[benzofuran-2,4′-cyclohex-2-ene]-1′,3-dione;
(2S,5′R)-7-chloro-3′,4-dimethoxy-5′-methyl-6-(3-pyridyl) spiro[benzofuran-2,4′-cyclohex-2-ene]-1′,3-dione; or
(2S,5′R)-7-chloro-3′,4,6-trimethoxy-5′-methyl-spiro[benzofuran-2,4′-cyclohex-2-ene]-1′,3-dione.

24. (canceled)

25. The method of claim 23, wherein the compound is (2S,5′R)-7-chloro-3′,4-dimethoxy-5′-methyl-6-(5-methyl-1,3,4-oxadiazol-2-yl) spiro[benzofuran-2,4′-cyclohex-2-ene]-1′,3-dione or a pharmacologically acceptable salt thereof.

26-28. (canceled)
